(19) United States
(12) Patent Application Publication
(10) Pub. No.: US 2006/0036280 A1
(43) Pub. Date: Feb. 16, 2006

(54) Systems and methods for severing occlusive elements from a delivery catheter

(75) Inventors: Ronald French, Santa Clara, CA (US); Ryan K. Pierce, Mountain View, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Concentric Medical, Inc., Mountain View, CA (US)

(21) Appl. No.: 10/917,260

(22) Filed: Aug. 11, 2004

(51) Int. Cl. A61M 29/00

(52) U.S. Cl. 606/200

(57) ABSTRACT

The present invention advantageously provides alternative systems and methods for severing implantable occlusive devices, particularly hydratable polymeric filaments, from delivery catheters during and/or subsequent to intraluminal delivery, advancement, and/or positioning of occluding filaments into treatment sites. One method for severing an occlusive element from an intraluminal delivery catheter comprises positioning a delivery catheter in a body lumen and advancing at least one occlusive element to a target site. Energy is then transmitted to a severing element so as to break the occlusive element at a selected location along a length thereof for its release to the target site.

### BACKGROUND OF THE INVENTION

1. Field of the Invention

The present application relates generally to medical systems and methods. More particularly, the present application relates to systems and methods for severing implantable occlusive elements from intraluminal delivery catheters.

Numerous persons experience some form of hemorrhagic stroke or blood vessel rupture in the brain. Vaso-occlusive devices are surgical implements or implants that are placed within the vasculature of the human body, typically via a catheter, either to block the flow of blood through a vessel making up that portion of the vasculature by formation of an embolus or to form such an embolus within an aneurysm stemming from the vessel. Other vascular abnormalities treated using such devices include arterio-venous malformations, fistulas, and burst blood vessels. Significantly, abnormal vasculature generated in the process of tumor growth may be treated using these vaso-occlusive devices.

The use of such devices has grown radically outside the use of treatment of the vasculature. Virtually any anatomical fluid vessel or opening has been treated or closed using devices of this type.

There are a variety of materials and vaso-occlusive devices commercially and medically in use. Perhaps the most well known of these devices is the Guglielmi Detachable Coil (GDC) shown in U.S. Pat. Nos. 5,122,136 and 5,354,295, both to Guglielmi et al. These patents and many more that follow it, describe a helically wound coil that is introduced to a treatment site in the body by use of a pusher wire that resembles a standard guide wire. The junction between the pusher wire and the coil is an electrolytically erodible joint that, upon application of a small current, will harmlessly erode in the human body separating the pusher wire from the coil. In overall summary, the procedure utilizing the GDC is this: the coil portion of the device is delivered by a catheter to the treatment site, the electricity is applied, the joint separates, the coil remains in the body forming the desired embolus, and the pusher wire and catheter are retrieved from the body. Many other variations of metallic coils are found in the patent literature and on the commercial marketplace. However, such coils are not entirely successful in achieving complete occlusion. For example, coil stiffness or coil pitch may leave voids at the treatment site resulting in recanalization, requiring follow-up procedures.

Another type of occluding material are embolic agents that are introduced into the human body in a liquid form where they are transformed either by precipitation from solution (e.g., U.S. Pat. No. 5,925,683 to Park) or by chemical reaction.

Another, more recently developed vaso-occlusive material involves biocompatible polymeric agents that are hydratable or gels. They may be introduced into treatment sites in the body much in the same way that the coils are although they typically must be handled in a somewhat different fashion because of the nature of their makeup. The polymers typically are quite slippery and may be damaged if handled with lack of care and understanding. In particular, severing occlusive elements from delivery catheters during and/or subsequent to intraluminal delivery, advancement, and/or positioning of vaso-occluding materials into treatment sites requires careful handling. Current devices and methods typically employ mechanical or chemical mechanisms to permit severing or detaching the occlusive element for its release at the target site. Alternative intraluminal severing devices and methods would further be advantageous.

2. Description of the Background Art

Endovascular therapies for treating vessel ruptures and blood flow abnormalities include implanting vaso-occlusive agents, coils and other devices such as that described in U.S. Pat. No. 4,994,069, and delivering hydrogel vaso-occluding particles and filaments into the vessels to be treated, as described in U.S. Patent Application Publication Nos. 2002/0193812A1; 2002/0193813A1; 2003/004533A1; 2003/0004568A1; and 2002/0193812A1, as well as U.S. patent application Ser. Nos. 10/400,185; 10/739,900; and 10/873,408, each of which are assigned to the assignee of the present application. U.S. Pat. Nos. 5,122,136; 5,354,295; and 5,925,683 have been described above. U.S. Pat. No. 6,299,590 describes methods and devices for inserting a ball-shaped implant made from pliable fibers. U.S. Pat. No. 6,312,421 describes the delivery of a biocompatible polymeric string to an aneurysm where the string is cut when the aneurysm is substantially filled.

The full disclosures of each of the above mentioned references are incorporated herein by reference in their entirety.

### BRIEF SUMMARY OF THE INVENTION

The present invention advantageously provides alternative systems and methods for severing implantable occlusive devices, particularly hydratable polymeric filaments, from delivery catheters during and/or subsequent to intraluminal delivery, advancement, and/or positioning of occluding filaments into treatment sites. The devices and methods of the present invention provide a variety of mechanisms to permit severing or detaching the occlusive element for its release at the target site. It is to be understood that the occluding filaments described herein are not limited to occluding blood vessels or aneurysms. Rather, the occluding elements described herein may be used to form an occlusion in any of the vessels, ducts, and cavities found in the body including but not limited to vessels found in the blood vasculature.

In a first aspect of the present invention, a method for severing an occlusive element from an intraluminal delivery catheter or sheath is provided. A delivery catheter is positioned in a body lumen, such as a blood vessel, and at least one occlusive element is advanced to a target site, which may comprise an aneurysm, vasculature of a tumor, an arterio-venous malformation, fistula, or burst blood vessel. Energy is transmitted to a severing element so as to break the occlusive element at a selected location along a length thereof for its release to the target site. The selected location for severing will typically be determined by an amount needed to fill the target site.

The transmitted energy advantageously heats the severing element so as to improve the cutting action of the severing element. The severing element is typically heated to a temperature in a range from about body temperature to about 700° F., preferably in a range from about 200° F. to about 600° F. The breaking of the occlusive element is performed using the heated severing element. It will be appreciated that there may further be some residual heating of the occlusive element itself. Typically, the heated severing element will be pulled proximally, retracted, or wound so as to change a shape (e.g., diameter) of the severing element. Alternatively, heating may automatically change a length or shape of the severing element. In either case, the heated severing element generally traverses across an axis of the occlusive element to allow for its detachment and release to the target site.

The transmitted energy preferably comprises electrical current. Electrical energy may be supplied with a controlled voltage power supply, a controlled current power supply, or a power supply employing both voltage and current control. In other embodiments, the energy transmitted may comprise radiofrequency energy, microwave energy, ultrasound energy, and like energy modalities that can be used to heat the severing element. The energy source is adapted for both bipolar and monopolar transmission. Bipolar instruments are typically connected to both poles of an energy source, wherein the energy flow is typically limited to the working end of the bipolar instrument (e.g., distal end of severing element). Monopolar devices are typically used in conjunction with a grounding pad wherein one pole of the energy source is coupled to the instrument and the other pole is coupled to the grounding pad. The energy flow in monopolar devices travels from the instrument (e.g., severing element) to the grounding pad. Energy transmission may be carried out before and/or during breaking the occlusive element.

The breaking is generally performed at a distal end of the delivery catheter once a desired amount of occlusive element is advanced into the target site. Advancement of the occlusive element may comprise pulling, pushing, or injecting the occlusive element to the target site. The method further comprises hydrating the filament, generally prior to advancing and/or breaking the occlusive element, so as to form a polymeric gel.

In another aspect of the present invention, a system for severing an occlusive element from an intraluminal delivery catheter is provided. The system generally includes a delivery catheter or sheath, an occlusive element severing device disposed within the catheter, and an energy source in communication with the severing device. The severing device may comprise a variety of mechanical mechanisms, such as a constricting loop, wire noose, tensioning wire, looped wire, spring wire, hook, and the like. Additional cutting structures that may be modified for use with the present invention are discussed in more detail in U.S. patent application Ser. Nos. 10/400,185 and 10/739,900, assigned to the assignee of the present application.

Generally, the severing wire comprises superelastic metal or shape memory alloy (e.g., NITINOL®, nickel titanium) or other conductive materials (e.g., stainless steel). A proximal portion of the severing wire may be insulated with a material, such as parylene, which minimizes heat transmission to structures other than the severing element, such as the catheter wall. Additionally, or alternatively, the proximal portion of the severing wire may be coated with a conductive material, such as gold, so that heat transmission is more focused at a distal end of the severing device. A cutting zone of the severing wire at the distal end will be initially larger than a diameter of the occluding element and smaller than a diameter of the delivery catheter.

The delivery catheter or sheath may be formed from a variety of medical grade materials, such as polymer tubes. In some instances, the polymer tubes may be reinforced with a braided metal or polymer, and/or lined with a low-friction material such as PTFE (polytetrafluoroethylene) or polyethylene, and/or coated with low-friction, hydrophilic coatings, such as hyaluronan-based coatings (e.g., HYDAK®) or polyvinylpyrrolidone-based coatings. The delivery catheter will generally have a length in a range from about 5 cm to about 300 cm, preferably from about 50 cm to about 250 cm, and a diameter in a range from about 0.018 inch to about 0.375 inch, preferably from about 0.030 inch to about 0.100 inch. The severing device may at least be partially integrated into a wall of the catheter, for example through a lumen separate of the occluding element lumen. Still further, the severing device may be partially integrated into a braided structure of the catheter. This catheter integration may further act to provide additional insulation to a proximal portion of the severing device.

The energy source may comprise an electrical conductor, battery, or generator. For example, a simple battery that supplies a controlled voltage power or a controlled current power may transmit electrical energy so as to heat the severing device. Such a battery operated system is convenient and cost effective. Optionally, the energy source may transmit alternative forms of energy, such as radiofrequency energy, microwave energy, ultrasound energy, and the like, so as to heat the severing element. As described above, the energy source further comprises bipolar and/or monopolar connections.

The system may further include an occlusive element disposed within the delivery catheter, the composition of which is discussed in more detail in U.S. Patent Application Publication Nos. 2002/0193812A1 and 2002/0193813A1, assigned to the assignee of the present application, generally comprises a polymeric filament that is hydratable so as to form a polymeric gel. The occlusive element may form a final hydrated noodle shape having a length of at least about 0.5 cm, preferably in a range from about 2 cm to about 200 cm, and a diameter in a range from about 0.004 inch to about 0.125 inch, preferably from about 0.005 inch to about 0.025 inch. It is to be understood that the occlusive element may also take on a variety of other filament and particle shapes, sizes, and forms. It will further be appreciated that additional mechanisms or features may be built into the structure of the occlusive element itself that will cooperate in some fashion to cause or to permit severing or detaching the occlusive element for its release at the target site.

In a further aspect of the present invention, a method for severing an occlusive element from an intraluminal delivery catheter is provided. A delivery catheter is positioned in a body lumen, such as a blood vessel, and at least one occlusive element is advanced to a target site, which may comprise an aneurysm, vasculature of a tumor, an arterio-venous malformation, fistula, or burst blood vessel. This method further includes winding a looped severing element so as to break the occlusive element at a selected location along a length thereof for its release to the target site. The selected location for severing will typically be determined by an amount needed to fill the target site.

In this embodiment, the winding is performed by rotating a mandrel so that the severing loop winds onto the mandrel. Winding generally reduces a diameter of the severing loop, which will be initially larger than a diameter of the occluding element and smaller than a diameter of the delivery catheter. The breaking is performed using the looped severing element, wherein the severing element traverses across an axis of the occlusive element as it is wound onto the mandrel. The occlusive element is generally severed at a distal end of the delivery catheter. As discussed above, this looped severing element may additionally be heated so as to enhance its ability to cut through the occlusive element.

In a still further aspect of the present invention, a system for severing an occlusive element from an intraluminal delivery catheter is provided. The system generally includes a delivery catheter or sheath and an occlusive element severing device disposed within the catheter. The severing device may comprise a looped severing element wound on a mandrel. The looped severing element preferably comprises a looped wire formed from superelastic metal or shape memory alloy (e.g., NITINOL®, nickel titanium) or other conductive materials (e.g., stainless steel). Alternatively, the looped severing element may also be formed from other materials, such as polymer string. The mandrel preferably comprises a wire having a thickness that is larger than that of the severing element wire. The mandrel may be formed from materials similar to those described above with respect to the severing element. Generally, the severing device is at least partially disposed within a first lumen of the catheter, while the occlusive element is disposed within a second lumen of the catheter. Further, this system may further incorporate an energy source in communication with the severing device.

In yet another aspect of the present invention, a method for severing an occlusive element from an intraluminal delivery catheter is provided. A delivery catheter is positioned in a body lumen, such as a blood vessel, and at least one occlusive element is advanced to a target site, which may comprise an aneurysm, vasculature of a tumor, an arterio-venous malformation, fistula, or burst blood vessel. This method further includes inflating a pair of cuffs having axially offset ridges so as to break the occlusive element at a selected location along a length thereof for its release to the target site. The selected location for severing will typically be determined by an amount needed to fill the target site. Severing is performed by pinching the occlusive element at the selected location between the axially offset ridges located at a distal end to the delivery catheter. The inflated cuffs may also serve as a safety valve to prevent further noodle advancement.

In still another aspect of the present invention, a system for severing an occlusive element from an intraluminal delivery catheter is provided. The system generally includes a delivery catheter or sheath and an occlusive element severing device disposed within the catheter. The severing device may comprise a pair of inflatable cuffs having ridges that are axially offset. The inflatable cuffs, which may optionally be separate or formed integrally with an inner sleeve of the delivery catheter, may comprise elastomeric materials, such as silicone rubber, latex rubber, and the like. In some embodiments, the axially offset ridges may form sharpened peaks.

A further understanding of the nature and advantages of the present invention will become apparent by reference to the remaining portions of the specification and drawings.

### BRIEF DESCRIPTION OF THE DRAWINGS

The following drawings should be read with reference to the detailed description. Like numbers in different drawings refer to like elements. The drawings, which are not necessarily to scale, illustratively depict embodiments of the present invention and are not intended to limit the scope of the invention.

FIG. 1 illustrates a perspective view of a typical catheter assembly containing a an occlusive element sticking out from one end.

FIG. 2 illustrates a partial cutaway of introduction of an occlusive implant into an aneurysm in the vasculature using a catheter.

FIG. 3 illustrates a side view of a delivery catheter containing a severing element constructed in accordance with the principles of the present invention.

FIGS. 4A through 4C illustrate cross sectional views of a distal end of the severing catheter of FIG. 3.

FIG. 5 illustrates a side view of another embodiment of the delivery catheter containing a severing element constructed in accordance with the principles of the present invention.

FIGS. 6A through 6C illustrate cross sectional views of a distal end of the severing catheter of FIG. 5.

FIGS. 7A and 7B illustrate side views of yet another embodiment of the delivery catheter containing a severing element constructed in accordance with the principles of the present invention.

FIGS. 8A through 8C illustrate side views of a further embodiment of the delivery catheter containing a severing element constructed in accordance with the principles of the present invention.

FIGS. 9A through 9C illustrate cross sectional views of a distal end of the severing catheter of FIGS. 8A through 8C respectively.

FIGS. 10A and 10B illustrate side views of a still further embodiment of the delivery catheter containing a severing element constructed in accordance with the principles of the present invention.

FIGS. 11A and 11B illustrate side views of another embodiment of the delivery catheter containing a severing element constructed in accordance with the principles of the present invention.

FIGS. 12A and 12B illustrate a method for severing an occlusive element during intraluminal delivery of the occlusive element into an aneurysm using the delivery catheter of FIG. 3.

### DETAILED DESCRIPTION OF THE INVENTION

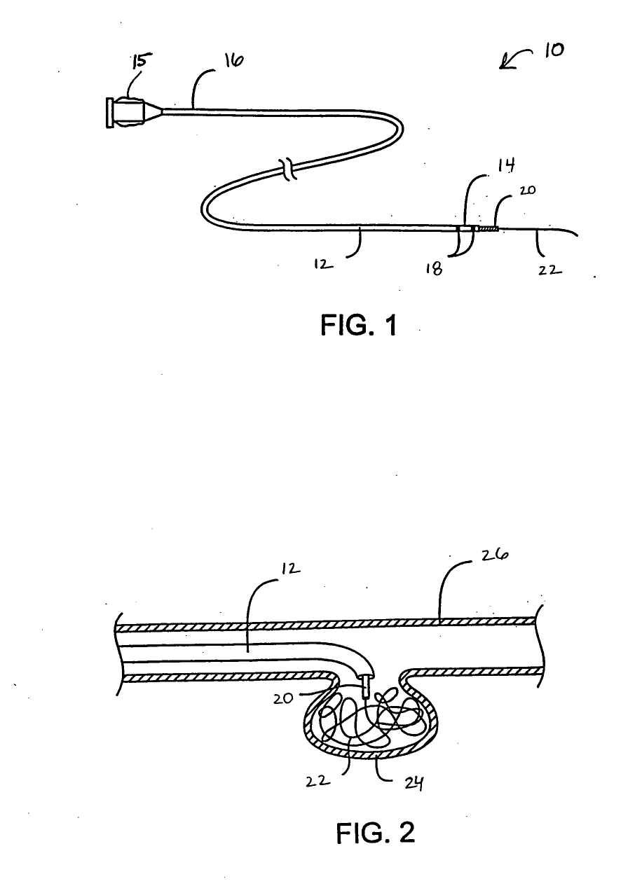

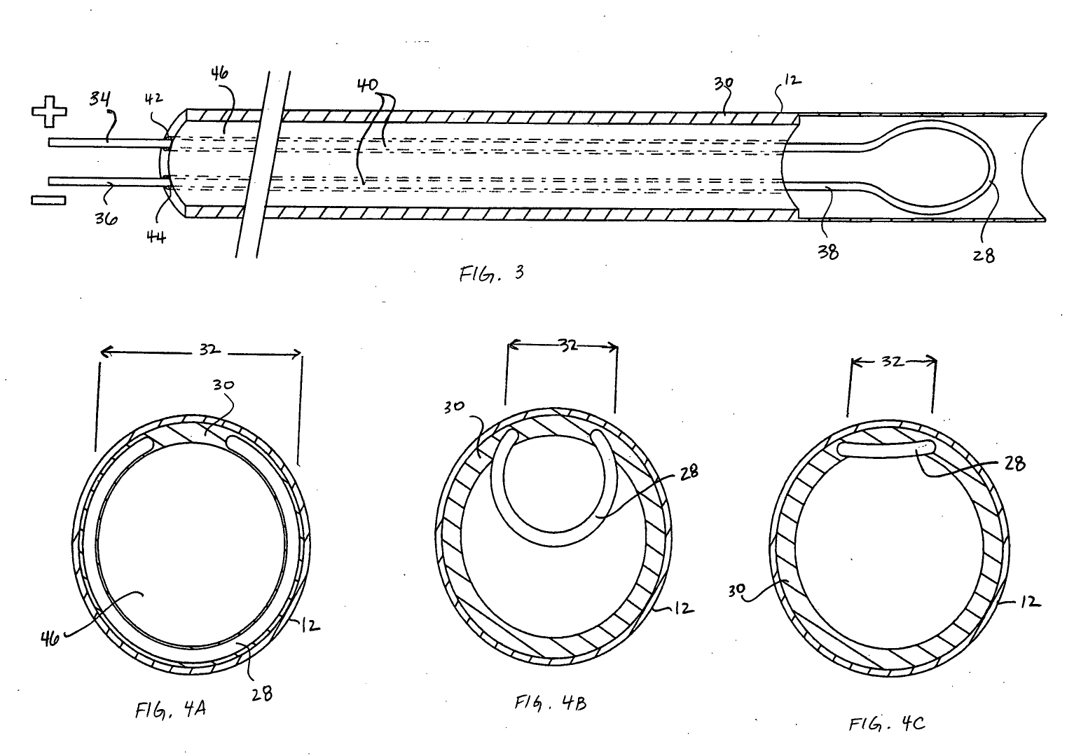

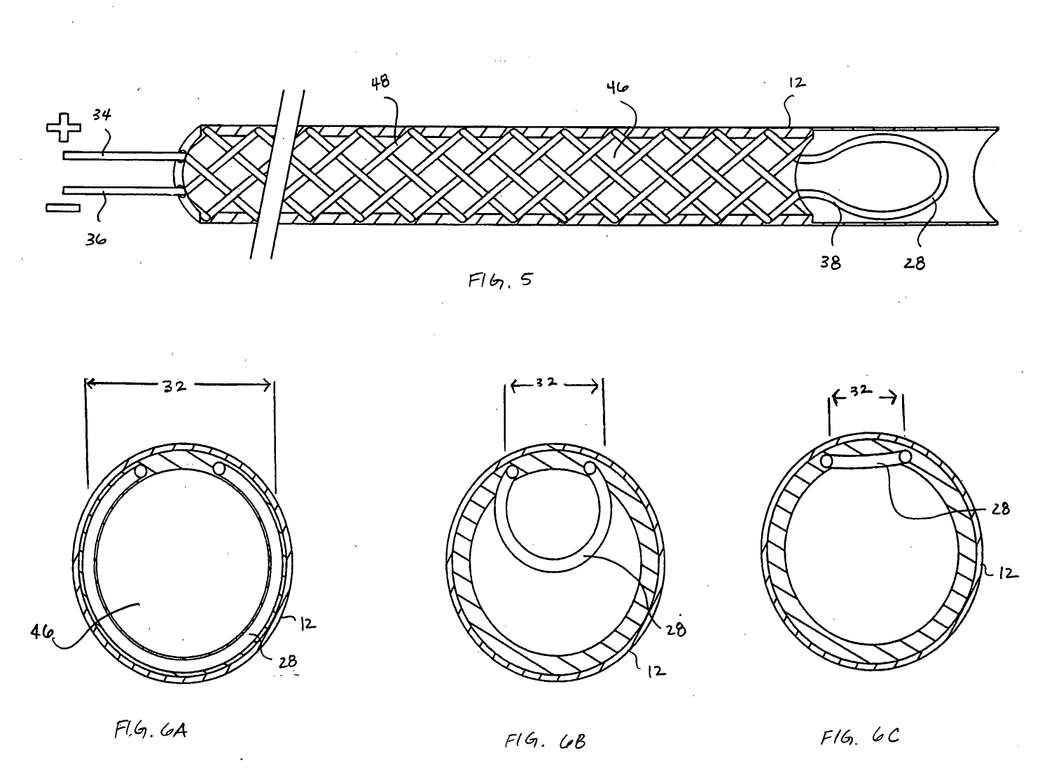

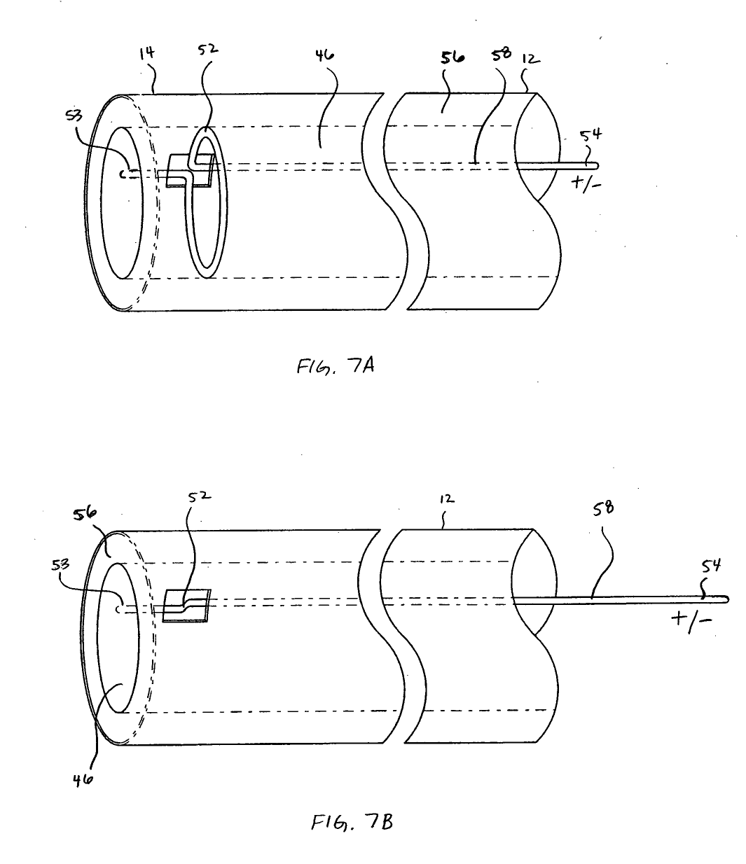

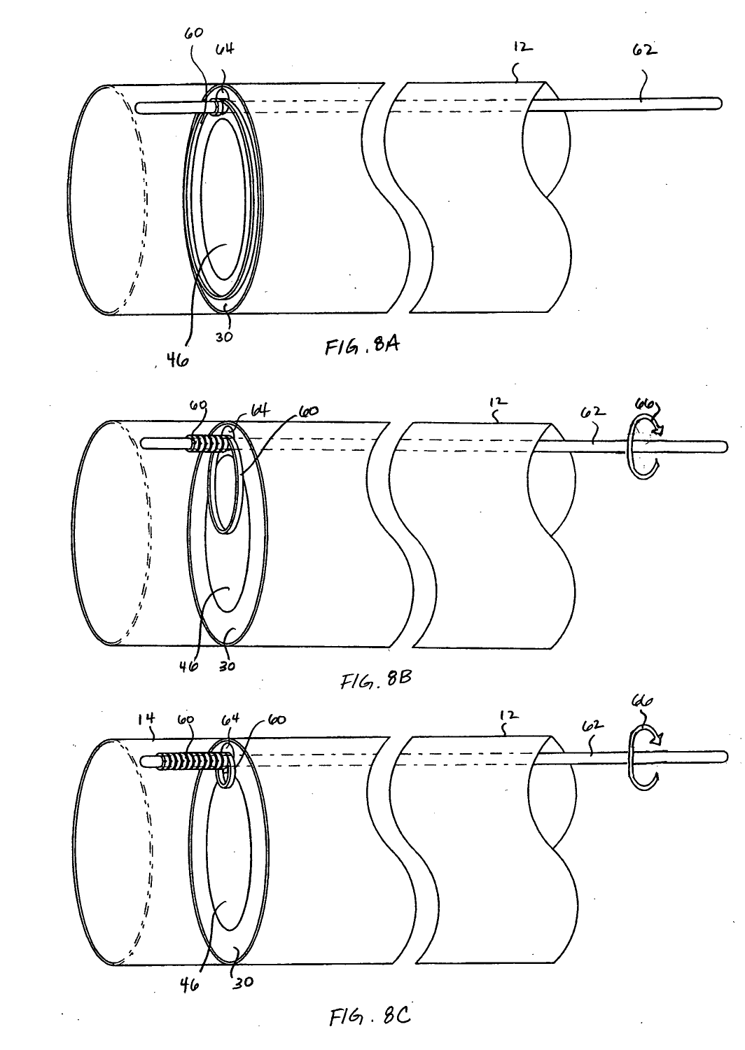

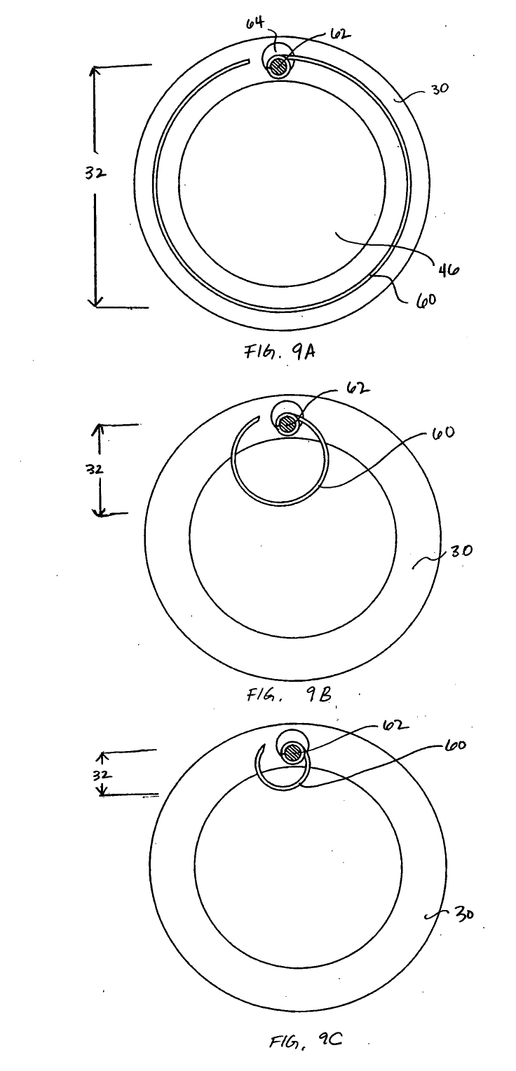

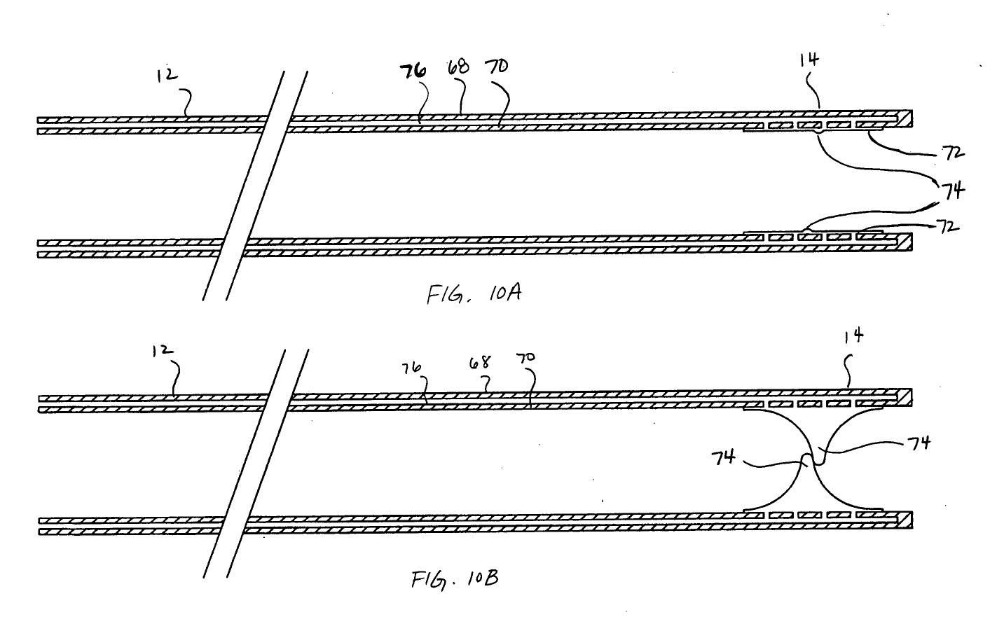

Typically, the occlusive device or element described herein will be delivered using a catheter assembly, e.g. (10) as shown in FIG. 1. Catheters are well known devices for delivering occlusive devices into the vasculature. They are thoroughly designed and many variations are available for reaching various regions in the vasculature whether the selected site for treatment be in a large vessel such as the descending aorta or in the fine and narrow vasculature of the brain. Shown in FIG. 1 is a catheter (12) that often is constructed in such a way that the distal end (14) of the catheter (12) is significantly less stiff than the proximal end (16). When the catheter (12) is small, e.g., because it is to be used in the neurovasculature, this is especially true. The proximal construction of the assembly generally includes a conventional hub (15) coupled to the proximal end (16). Also shown in FIG. 1 are radio-opaque markers (18) that allow the end of the catheter to be readily observed using fluoroscopy. The delivery sheath (20) is also shown as is the filamentary occlusion device (22).

FIG. 2 shows the placement of a catheter (12) such as that shown in FIG. 1 as it is used in providing a pathway for the delivery sheath (20) and the occluding element (22). In FIG. 2, the occlusive element (22) is used to fill an aneurysm (24) that extends from a parent vessel (26). It will be appreciated that the above depictions are for illustrative purposes only and do not necessarily reflect the actual shape, size, or dimensions of the system (10). This applies to all depictions hereinafter.

This system may deliver one or more occlusive elements. Typically, the occlusive element will comprise filamentary shapes. Of particular interest are filaments comprising natural or synthetic polymeric hydratable gel. Synthetic polymers may be, for instance, selected from the group consisting of polyacrylamide (PAAM), hydrophilic polyacrylonitrile (HYPAN), poly (N-isopropylacrylamine) (PNIPAM), poly (vinylmethylether), poly (ethylene oxide), poly (vinylalcohol), poly (ethyl (hydroxyethyl) cellulose), poly(2-ethyl oxazoline), polylactide (PLA), polyglycolide (PGA), poly(lactide-co-glycolide) PLGA, poly(ε-caprolactone), polydiaoxanone, polyanhydride, trimethylene carbonate, poly((\ddot{γ}-hydroxybutyrate), poly(g-ethyl glutamate), poly(DTH-iminocarbonate), poly(bisphenol-A iminocarbonate), poly(orthoester) (POE), polycyanoacrylate (PCA), polyphosphazene, polyethylene oxide (PEO), polyethyleneglycol (PEG), polyacrylic acid (PAA), polyacrylonitrile (PAN), polyvinylacrylate (PVA), polyvinylpyrrolidone (PVP), polyglycolic lactic acid (PGLA), their block and random copolymers, and their blends. Natural polymers, for instance, may be materials selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin, keratin, pectin, elastin, and their block and random copolymers and their blends. In addition, the occlusive elements may contain or be coated with one or more bioactive agents in an amount effective to provide or to promote a selected biological activity and may contain one or more radio-opacifiers.

The bioactive agent typically is selected to provide or to promote a biological activity at the occlusive device's selected implantation site. For instance, the bioactive agent may be selected from the group consisting of compositions that occlude blood flow, adhere to the occlusive device at the site, rebuild damaged vascular wall, regress or inhibit capillary dilation, regress or inhibit venous malformation, and regress or inhibit tumor growth at or near the implantation site.

By way of further example, the bioactive agent may be selected from the group consisting of protein factors, growth factors, inhibiting factors, endothelization factors, extracellular matrix-forming factors, cell adhesion factors, tissue adhesion factors, immunological factors, healing factors, vascular endothelial growth factors, scarring factors, tumor suppression antigen-binding factors, anti-cancer factors, monoclonal antibodies, monoclonal antibodies against a growth factor, drugs, drug producing cells, cell regeneration factors, progenitor cells of the same type as vascular tissue, and progenitor cells that are histologically different from vascular tissue.

The term “an effective amount of” a given agent or agents is to be determined on an agent-by-agent basis, taking into account such standard, known parameters of bioactive agents such as potency, available concentration, and volume of space within the patient to be targeted for the desired effect. Efficacy and proper dosage may be determined by routine assays specific for the bioactive agent selected using, for example, standard assays found in well known and frequently used laboratory assay and protocol manuals for identifying activity and quantifying potency of molecules and cells.

The occlusive elements may also comprise a radio-opacifier, e.g., a material that provides visibility of the device under X-ray or other imaging technology such as computer assisted tomography (CT scans), magnetic resonance imaging (MRI's), and fluoroscopy. For instance, a selected radio-opacifier may include a gadolinium based MRI contrast agent. These agents may include gadopentetate, gadopentetate dimeglumine (Gd-DTPA sold as “Magnevist”), gadoteridol (Gd HP-1303A sold as “ProHance”), gadodiamide (Gd-DTPA-BMA sold as “Omniscan”), gadoversetamide (Gd-DTPA-BMEA sold as “OptiMARK”), Gd-DOTA (sold as “Magnevist” or “lotarem”), Gd-DTPA labeled albumin, and Gd-DTPA labeled dextran. Other suitable fluoroscopic radio-opacifiers include those that are variously soluble in the polymer precursors or the polymer itself, e.g., metrizamide (see, U.S. Pat. No. 3,701,771) or iopromide (see, U.S. Pat. No. 4,364,921—often sold in a dilute form under the tradename “Ultravist”) and solid, powdered materials such as barium sulfate, bismuth trioxide, bismuth carbonate, tungsten metal, and tantalum metal, and the like. Other iodine based and powdered metal-based radio-opacifiers are also well-known.

The bioactive agents and radio-opaque materials may be integrated into the typically extruded occlusive elements. Integration or inclusion of the bioactive agents and radio-opaque materials into the extruded product may be accomplished during extrusion or after extrusion. Such integration may be accomplished after extrusion such as by the acts consisting of coating, dipping, jacketing, spraying, weaving, braiding, spinning, ion implantation, vapor deposition, and plasma deposition. Integration of the bioactive agents and radio-opaque materials during extrusion may also be accomplished by placing the agent into a solvent used to dissolve the polymeric material making up the occluding filament. The bioactive agents and radio-opaque materials may (depending upon their composition) also be incorporated into the filament during subsequent hydration of the extruded filament. It will be appreciated that the composition of the occlusive element may vary along its length and may well have certain features built into the structure that will cooperate in some fashion to cause or to permit severing or detaching it.

Referring now to FIGS. 3 and 4A through 4C, a variety of mechanisms may be incorporated into the structure of the delivery catheter (12) or sheath (20) that will cooperate in some fashion to permit severing or detaching the occlusive element (22) for its release at the target site (24). In particular, the embodiment shown in these figures illustrates a delivery catheter (12) and an occlusive element severing device (28) disposed at least partially within a wall (30) of the catheter. The severing device in this embodiment comprises a wire constricting loop (28) formed from superelastic metal or shape memory alloy (e.g., NITINOL®, nickel titanium) or other conductive materials. Further, an energy source (not shown) typically coupled to the hub (15) at a proximal end (16) of the delivery catheter, is in communication with the wire noose (28) so as to provide bipolar energy transmission, as denoted by the positive (+) and negative (−) connections (34, 36).

In operation, bipolar energy is transmitted to the wire noose (28) so as to break the occlusive element at a selected location along a length thereof for its release to the target site. The transmitted energy advantageously heats the wire noose (28) so as to improve its cutting action. The breaking of the occlusive element is performed using the heated wire noose (28). Typically, the heated wire noose (28) at its proximal ends (40) will be retracted, as shown in FIGS. 4A through 4C, so as to reduce a shape (e.g., diameter (32)) of the wire noose (28) at a distal end (38) so that it traverses across an axis of the occlusive element to allow for its detachment and release to the target site. The cutting zone (32) of the wire noose (28) at the distal (38) end will be initially larger than a diameter of the occluding element (22) and smaller than a diameter of the delivery catheter (12), as illustrated in FIG. 4A.

Preferably, proximal portions (40) of the wire noose (28) may be insulated with a material, such as parylene, which minimizes heat transmission to structures other than the severing element, such as the catheter wall (30). Additionally, or alternatively, the proximal portions (40) of the severing wire (28) may be coated with a conductive material, such as gold, so that heat transmission is more focused at the distal end (38) of the wire noose (28). Additionally, catheter integration of the wire noose (28) into lumens (42, 44) in the wall (30) of the catheter (12) separate from the occluding element lumen (46) may further act to provide enhanced insulation. The delivery catheter (12) may be formed from a variety of medical grade materials, such as polymer tubes. The delivery catheter (12) will generally have a length in a range from about 5 cm to about 300 cm, preferably from about 50 cm to about 250 cm, and a diameter in a range from about 0.018 inch to about 0.375 inch, preferably from about 0.030 inch to about 0.100 inch. It will be appreciated that the wire noose (28) at the distal end (38) remains disposed within the catheter (12) to prevent further undesirable or unwanted heating of surrounding structures.

The energy source may comprise an electrical conductor, battery, or generator. For example, a simple battery that supplies a controlled voltage power or a controlled current power may transmit electrical energy so as to heat the wire noose (28). Such a battery operated system is convenient and cost effective. Optionally, the energy source may transmit alternative forms of energy, such as radiofrequency energy, microwave energy, ultrasound energy, and the like, so as to heat the severing element. As described above, the energy source further comprises bipolar and/or monopolar connections.

Referring now to FIGS. 5 and 6A through 6C, another embodiment of the delivery catheter (12) containing a wire noose (28) is illustrated. In this embodiment, the catheter polymer tube (12) is reinforced with a braided structure (48) formed from metal or polymer materials. The wire noose (28) is at least partially disposed within the braided structure (48) of the catheter (12) so as to provide an alternative conduction path. Integration of the wire noose (28) into the braided structure (48) of the catheter (12) separate from the occluding element lumen (46) may provide additional insulation to the proximal portions (40) of the wire noose (28). The wire noose (28) in this embodiment further comprises shape memory alloy (e.g., nickel titanium) so that upon energy transmission the shape memory characteristics are activated. As such, heating the wire noose (28) may change a shape (e.g., diameter (32)) or length of the severing element, as shown in FIGS. 6A through 6C. In this embodiment, heating reduces a shape (e.g., diameter (32)) of the wire noose (28) at a distal end (38) so that it traverses across an axis of the occlusive element to allow for its detachment and release to the target site.

Referring now to FIGS. 7A and 7B, still another embodiment of the severing catheter (12) is illustrated. In this embodiment, the severing device comprises a tensioning wire (52) formed from superelastic metal or shape memory alloy (e.g., NITINOL®, nickel titanium) or other conductive materials and having a distal end (53) which is anchored to a distal end (14) of the catheter (12). Further, an energy source (not shown) typically coupled to the hub (15) at a proximal end (16) of the delivery catheter, is in communication with the tensioning wire (52) so as to provide monopolar energy transmission, as denoted by the positive (+) or negative (−) connection (54). The tensioning wire (52) is at least partially disposed within a lumen (56) separate from the occluding element lumen (46) so as to provide additional insulation to a proximal portion (58) of the tensioning wire (52). The breaking of the occlusive element is performed using the heated tensioning wire (52). Typically, the heated tensioning wire (52) at the proximal end (58) will be retracted, as shown in FIG. 7B, so as to reduce a diameter of the tensioning wire (52) so that it traverses across an axis of the occlusive element to allow for its detachment and release to the target site. The distal anchoring of the tensioning wire (52) provides adequate tensile force application so as to allow severing of the occlusive element. It will be appreciated that this tensioning wire (52) embodiment can include, but is not limited to, heating of the cutting wire and may be formed from other materials, such as polymers including nylon, polyurethane, polyimide, polyester, polypropylene, polyethylene, silk, PTFE (polytetrafluoroethylene), ePTFE (expanded polytetrafluoroethylene), PET (polyethyleneterephthalate), CRISTAMID®, GRILAMID®, PEBAX®, and like threads or materials.

Referring now to FIGS. 8A through 8C and 9A though 9C, yet another embodiment of the severing catheter (12) is illustrated. In this embodiment, the severing device comprises a looped severing element (60) anchored on one end to a mandrel (62). The looped severing element (60) preferably comprises a looped wire formed from superelastic metal or shape memory alloy (e.g., NITINOL®, nickel titanium) or other conductive materials (e.g., stainless steel). Alternatively, the looped severing element (60) may be formed from other materials, such as polymers including nylon, polyurethane, polyimide, polyester, polypropylene, polyethylene, silk, PTFE (polytetrafluoroethylene), ePTFE (expanded polytetrafluoroethylene), PET (polyethyleneterephthalate), CRISTAMID®, GRILAMID®, PEBAX®, and like threads or materials. The mandrel (62) preferably comprises a wire having a thickness that is larger than that of the severing element wire (60). The mandrel (62) may be formed from materials similar to those described above with respect to the severing element. Generally, the severing device (60, 62) is at least partially disposed within a first lumen (64) in the wall (30) of the catheter (12), while the occlusive element is disposed within a second lumen (46) of the catheter (12).

In operation, the looped severing element (60) is wound on the mandrel (62) so as to break the occlusive element at a selected location along a length thereof for its release to the target site. The selected location for severing will typically be determined by an amount needed to fill the target site. Winding is generally performed by rotating the mandrel (62), as denoted by arrow 66 in FIGS. 8B and 8C, so that the severing loop (60) winds onto the mandrel in a “cord” and “spool” fashion. As illustrated in FIGS. 9A through 9C, winding generally reduces a diameter (32) of the severing loop (60). The cutting zone (32) of the severing loop (60) will be initially larger than a diameter of the occluding element (22) and smaller than a diameter of the delivery catheter (12), as shown in FIG. 9A. The breaking is performed using the looped severing element, wherein the looped wire (60) traverses across an axis of the occlusive element (22) as it is wound onto the mandrel (62). The occlusive element (22) is generally severed at a distal end (14) of the delivery catheter (12). Upon filament detachment, the wire noose (28) may be returned to its initial position (FIGS. 8A and 9A).

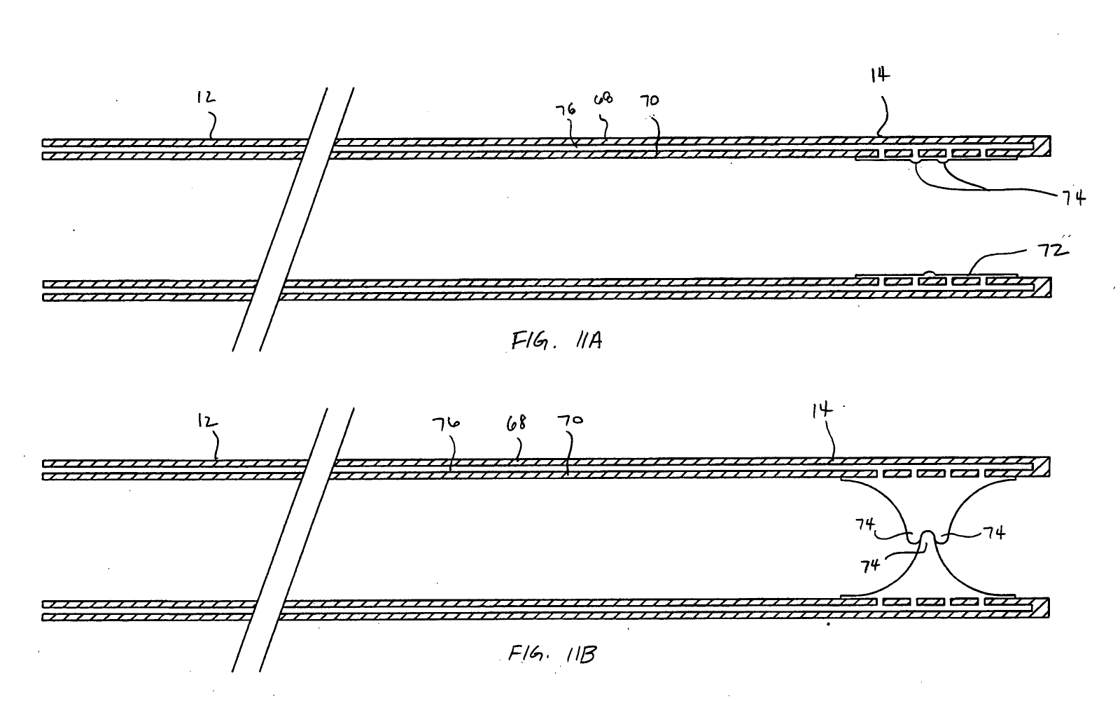

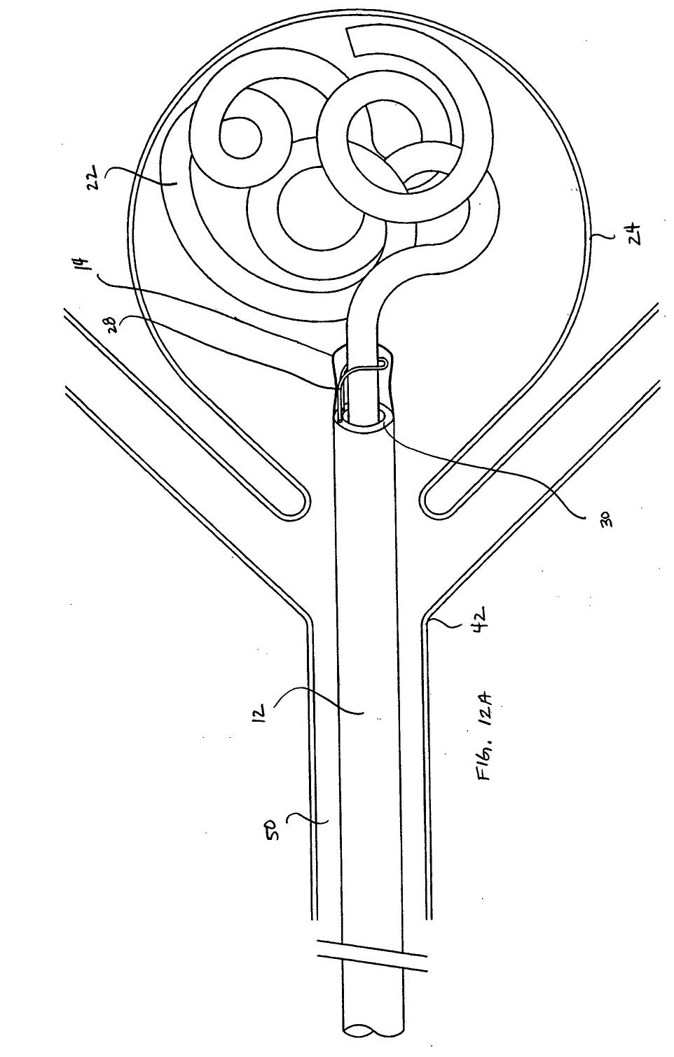

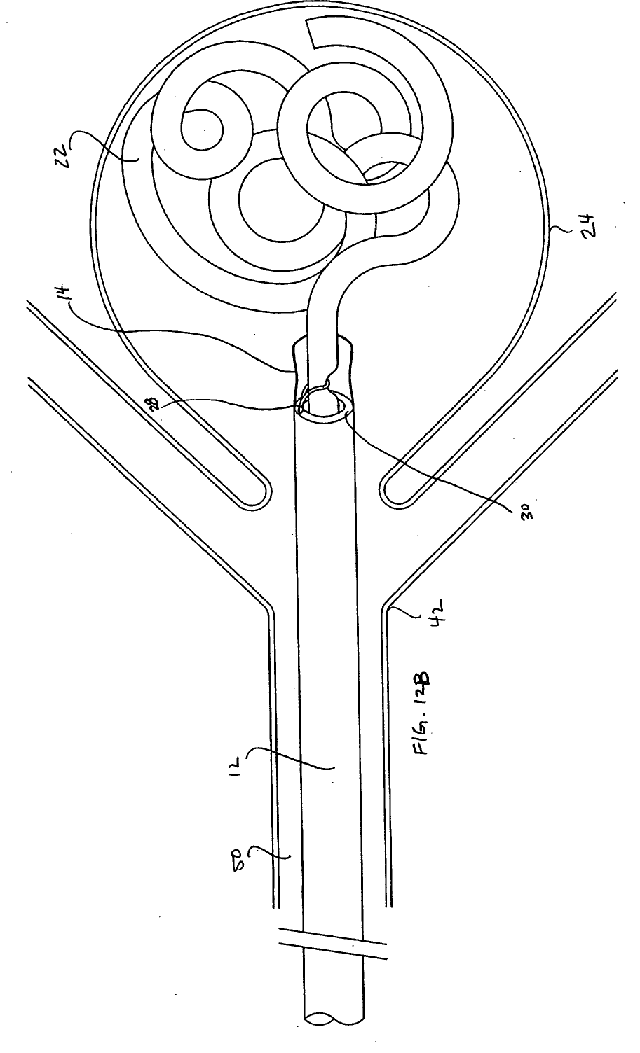

Referring now to FIGS. 10A, 10B, 11A, and 11B, further embodiments of the severing catheter (12) are illustrated. The catheter (12) in this embodiment comprises an outer sleeve (68) and an inner sleeve (70). The inner sleeve (70) includes a region in the form of inflatable cuffs (72). Axially offset ridges (74) are additionally formed over the inflatable cuffs (72). The sleeves (68) and (70) are mounted coaxially and define an annular inflation lumen (76) therebetween in communication with an inflation assembly (not shown) coupled to the hub (15) at a proximal end (16) of the delivery catheter. Thus, the inflatable cuffs (72) may be pressurized and inflated through the annular inflation lumen (76) (e.g., with fluid) to close radially inward, as shown in FIGS. 10B and 11B. In this way, an occlusive filamentary component (22) may be compressively deformed or pinched between the axially offset ridges (74) at the distal end (14) of the catheter (12) so as to break the occlusive element (22) at a selected location along a length thereof for its release to the target site (24). Additionally, the inflated cuffs (72) may serve as a safety valve to prevent further noodle (22) advancement when inflated.

The cutting geometry illustrated in FIGS. 10A and 10B shows two sharpened peaks (74), while the cutting geometry illustrated in FIGS. 11A and 11B shows three sharpened peaks (74). The inflatable cuffs (72) may be formed integrally with the inner sleeve (70), e.g., being a thinned or otherwise shaped region capable of being inflated to radially expand in an inward direction. Alternatively, the inflatable cuffs (72) may be made from a different material, such as an elastomeric material, e.g., silicone rubber, latex rubber, or the like. The axially offset ridges (74) may also be formed as an integral portion of the cuffs (74). Alternatively, it could comprise an array of axially offset ridges (74) which are attached to the inner surfaces of the inflatable cuffs (72).

Referring now to FIGS. 12A and 12B, a method for severing an occlusive element (22) from an intraluminal delivery catheter (12) during advancement of the filament (22) into an aneurysm (24) at a bifurcated vessel juncture (42) using the severing catheter of FIG. 3 is described. It will be appreciated that the present invention may be used in a variety of vessels, ducts, and cavities found in the body, and is not limited to bifurcated aneurysms. The delivery catheter (12) is positioned in a body lumen (50) with a distal end (12) thereof at the aneurysm (24) site at the bifurcated juncture (42). Once properly positioned, the occlusive filament (22) is advanced to the aneurysm (24). Energy is then transmitted to the wire noose (28) so as to break the occlusive element (22) at a selected location along a length thereof for its release to the aneurysm (24), as shown in FIG. 12B. The selected location for severing will typically be determined by an amount needed to fill the aneurysm (24).

The transmitted energy advantageously heats the wire noose (28) so as to improve its cutting action. The wire noose (28) is typically heated to a temperature in a range from about body temperature to about 700° F., preferably in a range from about 200° F. to about 600° F. The breaking of the occlusive element (22) is performed using the heated wire noose (28). Typically, the heated wire noose (28) at its proximal ends (40) will be retracted so as to reduce a diameter (32) of the wire noose (28) so that it traverses across an axis of the occlusive element (22) to allow for its detachment and release. Further, heating may enhance the ability of the wire noose (28) to cut through additional components associated with the occluding element (22), such as reinforcing members or sheaths as described in U.S. patent application Ser. No. 10/873,408, assigned to the assignee of the present application, or core members as described in U.S. Patent Application Publication No. 2003/0004568A1, assigned to the assignee of the present application. It will be appreciated that there may further be some residual heating and/or melting of the occlusive element (22) itself. The transmitted energy preferably comprises electrical current. Electrical energy may be supplied with a controlled voltage power supply, a controlled current power supply, or a power supply employing both voltage and current control. Further, the energy source may be adapted for both bipolar and monopolar transmission.

Energy transmission is typically carried out before and/or during breaking the occlusive element (22). The breaking is generally performed at a distal end (14) of the delivery catheter (12) once a desired amount of occlusive element (22) is advanced into the aneurysm (24). Advancement of the occlusive element (22) may comprise pulling, pushing, or injecting the occlusive element to the aneurysm (24). The method further includes hydrating the filament (22), generally prior to advancing and/or breaking the occlusive element (24), so as to form a polymeric gel. Upon filament detachment, the wire noose (28) may be returned to its initial position (FIG. 4A).

Although certain exemplary embodiments and methods have been described in some detail, for clarity of understanding and by way of example, it will be apparent from the foregoing disclosure to those skilled in the art that variations, modifications, changes, and adaptations of such embodiments and methods may be made without departing from the true spirit and scope of the invention. Therefore, the above description should not be taken as limiting the scope of the invention which is defined by the appended claims.

What is claimed is:

1. A method for severing an occlusive element from an intraluminal delivery catheter, the method comprising:
positioning a delivery catheter in a body lumen;
advancing at least one occlusive element to a target site;
transmitting energy to a severing element; and
breaking the occlusive element at a selected location along a length thereof for its release to the target site.

2. The method of claim 1, wherein the transmitted energy heats the severing element.

3. The method of claim 2, wherein the severing element is heated to a temperature in a range from about body temperature to about 700° F.

4. The method of claim 2, wherein the breaking is performed using the heated severing element.

5. The method of claim 4, wherein the severing element comprises a constricting loop.

6. The method of claim 4, wherein the severing element comprises a tensioning wire.

7. The method of claim 4, wherein the severing element comprises a looped wire.

8. The method of claim 4, wherein heating changes a length or shape of the severing element.

9. The method of claim 4, wherein the severing element traverses across an axis of the occlusive element.

10. The method of claim 1, wherein the transmitted energy comprises electrical current.

11. The method of claim 1, wherein the transmitted energy comprises radiofrequency energy, microwave energy, or ultrasound energy.

12. The method of claim 1, wherein the energy is transmitted in bipolar or monopolar operation.

13. The method of claim 1, wherein energy is transmitted before or during breaking the occlusive element.

14. The method of claim 1, wherein the breaking is performed at a distal end of the delivery catheter.

15. The method of claim 1, wherein advancing comprises pulling, pushing, or injecting the occlusive element to the target site.

16. The method of claim 1, wherein the target site comprises an aneurysm.

17. The method of claim 1, wherein the target site comprises vasculature of a tumor.

18. The method of claim 1, wherein the occlusive element comprises a filament.

19. The method of claim 18, further comprising hydrating the filament prior to breaking the occlusive element.

20. A system for severing an occlusive element from an intraluminal delivery catheter, the system comprising:
a delivery catheter;
an occlusive element severing device disposed within the delivery catheter; and
an energy source in communication with the severing device.

21. The system of claim 20, wherein the severing device comprises a wire constricting loop.

22. The system of claim 20, wherein the severing device comprises a tensioning wire.

23. The system of claim 20, wherein the severing device comprises a looped wire.

24. The system of claim 21, 22, or 23, wherein the wire comprises superelastic metal or shape memory alloy.

25. The system of claim 21, 22, or 23, wherein at least a proximal portion of the wire is insulated.

26. The system of claim 20, wherein the severing device is at least partially integrated into a wall of the delivery catheter.

27. The system of claim 20, wherein the severing device is at least partially integrated into a braided structure of the delivery catheter.

28. The system of claim 20, wherein the energy source comprises an electrical conductor, battery, or generator.

29. The system of claim 20, wherein the energy source transmits electrical current so as to heat the severing device.

30. The system of claim 20, wherein the energy source transmits radiofrequency energy, microwave energy, or ultrasound energy so as to heat the severing element.

31. The system of claim 20, wherein the energy source further comprises bipolar or monopolar connections.

32. The system of claim 20, further comprising an occlusive element disposed within the delivery catheter.

33. The system of claim 32, wherein the occlusive element comprises a filament that is hydratable.

34. The system of claim 33, wherein the filament comprises a polymeric gel.

35. A method for severing an occlusive element from an intraluminal delivery catheter, the method comprising:
positioning a delivery catheter in a body lumen;
advancing at least one occlusive element to a target site;
winding a looped severing element; and
breaking the occlusive element at a selected location along a length thereof for its release to the target site.

36. The method of claim 35, wherein winding is performed by rotating a mandrel so that the severing loop winds onto the mandrel.

37. The method of claim 35, wherein winding reduces a diameter of the severing loop.

38. The method of claim 37, wherein breaking is performed using the looped severing element.

39. The method of claim 38, wherein the severing element traverses across an axis of the occlusive element.

40. The method of claim 35, wherein the breaking is performed at a distal end of the delivery catheter.

41. The method of claim 35, wherein advancing comprises pulling, pushing, or injecting the occlusive element to the target site.

42. The method of claim 35, wherein the target site comprises an aneurysm.

43. The method of claim 35, wherein the target site comprises vasculature of a tumor.

44. The method of claim 35, wherein the occlusive element comprises a filament.

45. The method of claim 44, further comprising hydrating the filament prior to breaking the occlusive element.

46. The method of claim 35, further comprising heating the looped severing element.

47. A system for severing an occlusive element from an intraluminal delivery catheter, the system comprising:
a delivery catheter; and
an occlusive element severing device disposed within the delivery catheter, wherein the severing device comprises a looped wire wound on a mandrel.

48. The system of claim 47, wherein the looped wire comprises superelastic metal or shape memory alloy.

49. The system of claim 47, wherein the mandrel comprises a wire having a thickness that is larger than that of the looped wire.

50. The system of claim 47, wherein the severing device is at least partially disposed within a first lumen of the delivery catheter.

51. The system of claim 50, further comprising an occlusive element disposed within a second lumen of the delivery catheter.

52. The system of claim 51, wherein the occlusive element comprises a filament that is hydratable.

53. The system of claim 52, wherein the filament comprises a polymeric gel.

54. The system of claim 47, further comprising an energy source in communication with the severing device

55. A method for severing an occlusive element from an intraluminal delivery catheter, the method comprising:
positioning a delivery catheter in a body lumen;
advancing at least one occlusive element to a target site;
inflating a pair of cuffs having axially offset ridges; and
severing the occlusive element at a selected location along a length thereof for its release to the target site.

56. The method of claim 55, wherein the severing is performed by pinching the occlusive element at the selected location between the axially offset ridges.

57. The method of claim 55, wherein the inflated cuffs prevent further noodle advancement.

58. The method of claim 55, wherein the severing is performed at a distal end of the delivery catheter.

59. The method of claim 55, wherein advancing comprises pulling, pushing, or injecting the occlusive element to the target site.

60. The method of claim 55, wherein the target site comprises an aneurysm.

61. The method of claim 55, wherein the target site comprises vasculature of a tumor.

62. The method of claim 55, wherein the occlusive element comprises a filament.

63. The method of claim 62, further comprising hydrating the filament prior to severing the occlusive element.

64. A system for severing an occlusive element from an intraluminal delivery catheter, the system comprising:
a delivery catheter; and
an occlusive element severing device disposed within the delivery catheter, wherein the severing device comprises a pair of inflatable cuffs having ridges that are axially offset.

65. The system of claim 64, wherein the inflatable cuffs comprise elastomeric material.

66. The system of claim 64, wherein the inflatable cuffs are formed integrally with an inner sleeve of the delivery catheter.

67. The system of claim 64, wherein the axially offset ridges form sharpened peaks.

68. The system of claim 64, further comprising an occlusive element disposed within the delivery catheter.

69. The system of claim 68, wherein the occlusive element comprises a filament that is hydratable.

70. The system of claim 69, wherein the filament comprises a polymeric gel.
